(19) United States
(12) Patent Application Publication
(10) Pub. No.: US 2006/0004397 A1
(43) Pub. Date: Jan. 5, 2006

(54) Vitreous body cutter and vitreous body surgical equipment having the same

(75) Inventor: Koji Osawa, Anjo-shi (JP)

Correspondence Address: SUGHRUE MION, PLLC, 2100 PENNSYLVANIA AVENUE, N.W., SUITE 800, WASHINGTON, DC, 20037, US

(73) Assignee: NIDEK CO., LTD.

(21) Appl. No.: 11/171,500

(22) Filed: Jul. 1, 2005

(30) Foreign Application Priority Data
Jul. 1, 2004 (JP) P2004-195088

(51) Int. Cl. A61B 17/14

(52) U.S. Cl. 606/180

(57) ABSTRACT

A vitreous body cutter for cutting a vitreous body in an eye includes: an inner cylindrical blade having a first suction hole and a suction path; an outer cylindrical blade having a second suction hole, the outer cylindrical blade holding the inner cylindrical blade to be rotatable about a center axis thereof; a main body fixed with the outer cylindrical blade; a diaphragm arranged in the main body, the diaphragm being linearly advanced to a distal end side of the blades in a direction of the center axis by supplying compressed gas into a gas chamber formed by the diaphragm and the main body; and a converting-and-transmitting mechanism which converts linearly advancement of the diaphragm into a rotation about the center axis and transmits the rotation to the inner cylindrical blade to rotate the inner cylindrical blade about the center axis.

### BACKGROUND OF THE INVENTION

The present invention relates to a vitreous body cutter for cutting a vitreous body in an eye and a vitreous body surgical equipment (apparatus) having the same

In a vitreous body cutter used in vitreous body surgery, a vitreous body in an eye is drawn by suction (aspiration) through a suction (aspiration) hole formed in the vicinity of an extremity (a distal end) of an outer cylindrical (tubular) blade fixed to a cutter main body, to thus cause the vitreous body to fit into the suction hole. An inner cylindrical (tubular) blade is caused to linearly reciprocate in a direction of the center axis thereof with respect to the outer cylindrical blade (a guillotine type) (refer to U.S. Pat. No. 6,383,203 (JP-A-2001-87303)) or rotationally reciprocate about the center axis (a rotary type) (refer to U.S. Pat. No. 5,176,628 (JP-A-H03-207355)), to thus excise the fitted vitreous body.

Meanwhile, in the case of the guillotine type, the suction hole cannot be provided at the extremity of the outer cylindrical blade in view of a structure of the cutoff type and therefore, the vitreous body proximate to a retina is difficult to be cut (excited). In contrast thereto, in the case of the rotary type, the suction hole can be provided at the extremity of the outer cylindrical blade and therefore, the vitreous body proximate to the retina is easy to be cut (excited).

Two types of rotary structures are known; one is a structure in which the inner cylindrical blade is rotated in one direction by a motor included in the cutter main body, and the other is a structure in which the inner cylindrical blade is rotationally reciprocated by reciprocating a piston in a cylinder (chamber) included in the cutter main body by controlling and supplying compressed gas (refer to U.S. Pat. No. 5,176,628 (JP-A-3-207355)). However, the former is difficult to handle since the weight becomes heavy. Further, the vitreous body is easy to be involved between the outer cylindrical blade and the inner cylindrical blade by the one-directional rotation. On the other hand, the latter needs to overcome conflicting problems of reducing friction resistance between the piston and the cylinder and ensuring airtightness therebetween, conversion efficiency of converting pressure energy of the compressed gas into rotational energy is not excellent and a stable function is difficult to be ensured by a small drive source.

### SUMMARY OF THE INVENTION

In view of the problem of the related art, an object of the invention is to provide a vitreous body cutter of a rotary type capable of cutting a vitreous body stably even by a small drive source and a vitreous body surgical equipment (apparatus) having the same.

In order to resolve the above-described problem, the invention is characterized in providing the following constitution.

(1) A vitreous body cutter for cutting a vitreous body in an eye, the vitreous body cutter comprising:

an inner cylindrical blade having a first suction hole and a suction path;

an outer cylindrical blade having a second suction hole, the outer cylindrical blade holding the inner cylindrical blade to be rotatable about a center axis thereof;

a main body fixed with the outer cylindrical blade;

a diaphragm arranged in the main body, the diaphragm being linearly advanced to a distal end side of the blades in a direction of the center axis by supplying compressed gas into a gas chamber formed by the diaphragm and the main body; and

a converting-and-transmitting mechanism which converts linearly advancement of the diaphragm into a rotation about the center axis and transmits the rotation to the inner cylindrical blade to rotate the inner cylindrical blade about the center axis.

(2) The vitreous body cutter according to (1), wherein

the converting-and-transmitting mechanism includes a lead screw, a nut and a torsion spring, the lead screw and the nut being brought in mesh with each other,

one of the lead screw and the nut is fixed to the inner cylindrical blade,

the other of the lead screw and the nut is linearly advanced by the diaphragm being linearly advanced against spring force of the torsion spring by supplying the compressed gas into the gas chamber to rotate the one in a first direction, and

the other is linearly retreated by the diaphragm being linearly retreated by the spring force of the torsion spring by releasing the gas from the gas chamber to rotate the one in a second direction opposed to the first direction.

(3) The vitreous body cutter according to (1), wherein

the converting-and-transmitting mechanism includes a lead screw and a nut, the lead screw and the nut being brought in mesh with each other,

one of the lead screw and the nut is fixed to the inner cylindrical blade,

the other of the lead screw and the nut is linearly advanced by the diaphragm being linearly advanced by supplying the compressed gas into the gas chamber to rotate the one in a first direction, and

the other is linearly retreated by the diaphragm being linearly retreated by sucking the gas from the gas chamber to rotate the one in a second direction opposed to the first direction.

(4) The vitreous body cutter according to (1), wherein

the main body includes a blade portion fixed with the outer cylindrical blade holding the inner cylindrical blade, and a drive mechanism portion provided with the diaphragm and the converting-and-transmitting mechanism, and

the blade portion and the drive mechanism portion are separable from each other.

(5) The vitreous body cutter according to (4), further comprising a transmitting mechanism provided at the blade portion, the transmitting mechanism transmitting the rotation converted by the converting-and-transmitting mechanism to the inner cylindrical blade.

(6) A vitreous body surgical equipment comprising:

a vitreous body cutter according to claim 1;

a gas supply unit which supplies the compressed gas into the gas chamber of the vitreous body cutter;

a control unit which controls to supply the compressed gas; and

a suction unit which generates suction pressure in the suction path in the inner cylindrical blade.

### BRIEF DESCRIPTION OF THE DRAWINGS

FIGS. 1A and 1B are schematic side sectional views showing a state of fitting an inner cylindrical blade into an outer cylindrical blade.

FIGS. 2A and 2B are schematic front sectional views showing the state of fitting the inner cylindrical blade into the outer cylindrical blade.

FIG. 3 is a schematic constitution view of a vitreous body cutter and a vitreous body surgical equipment having the same.

FIG. 4 is a schematic constitution view showing a modified example of the vitreous body cutter.

### DETAILED DESCRIPTION OF PREFERRED EMBODIMENTS

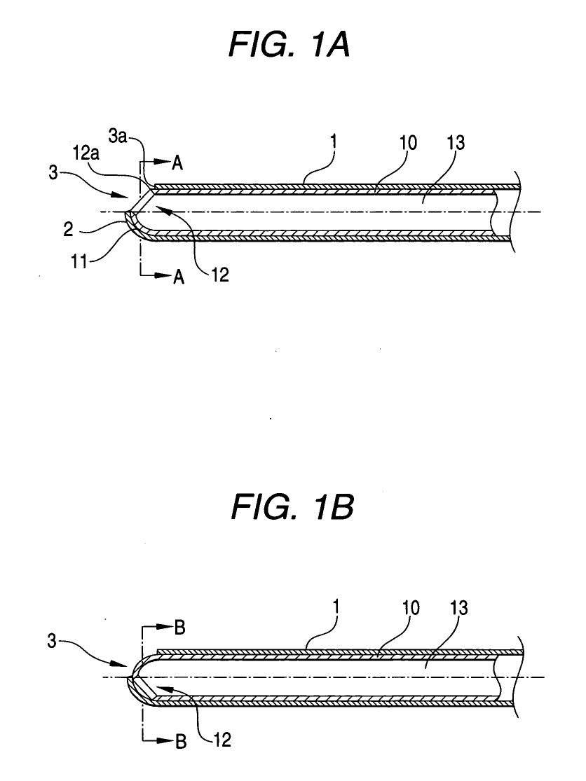

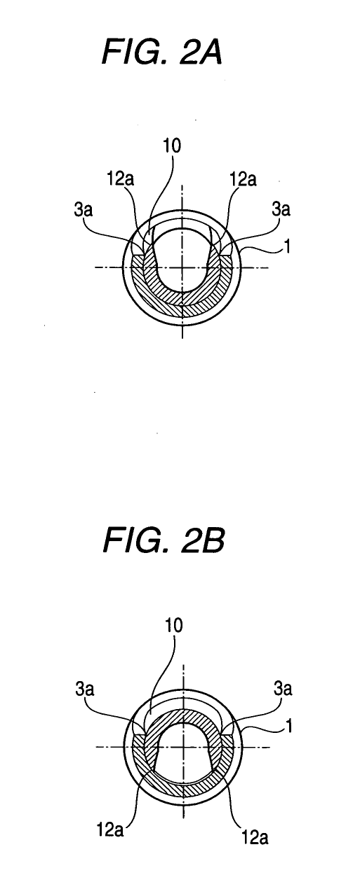

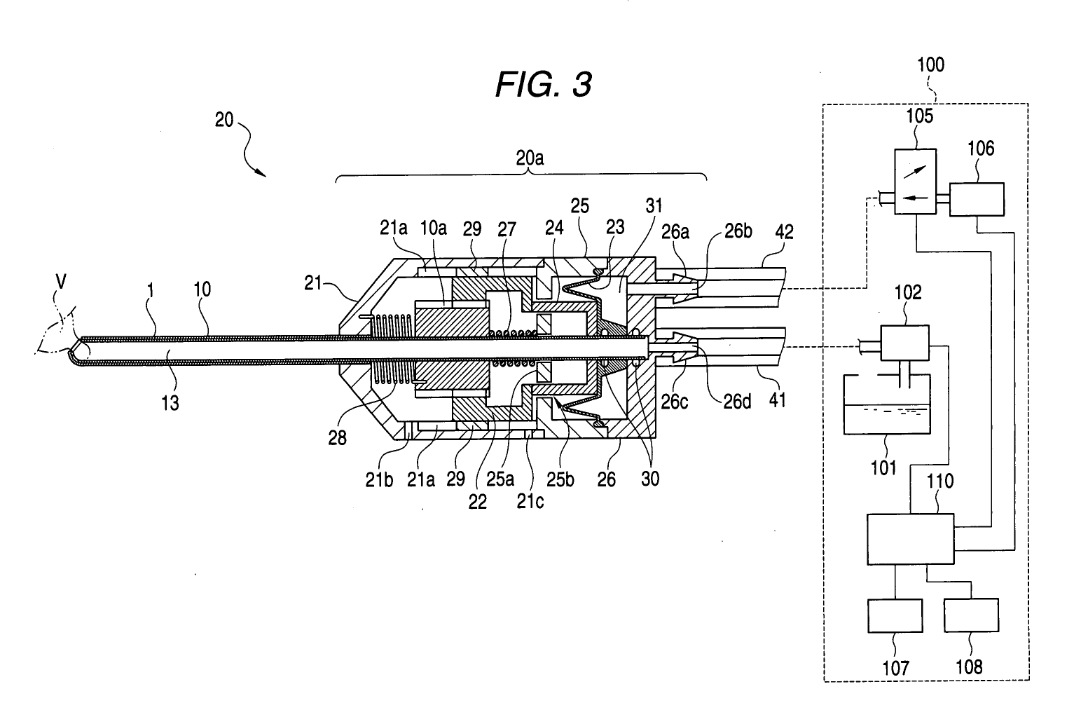

An embodiment of the invention will be explained in reference to the drawings. FIG. 3 is a schematic constitution view of a vitreous body cutter and a vitreous body surgical equipment having the same. FIGS. 1A and 1B are schematic side sectional views showing a state of fitting an inner cylindrical (tubular) blade into an outer cylindrical (tubular) blade. FIGS. 2A and 2B are schematic front sectional views showing the state of fitting the inner cylindrical blade into the outer cylindrical blade.

A vitreous body cutter 20 cuts (excises) a vitreous body V by rotating an inner cylindrical blade 10 with respect to an outer cylindrical blade 1 between a state where an opening 3 of the outer cylindrical blade 1 and an opening 12 of the inner cylindrical blade 10 overlap each other (an open state where the opening 12 can be viewed from the opening 3) (refer to FIG. 1A and FIG. 2A) and a state where the opening 3 and the opening 12 do not overlap each other (a closed state in which the opening 12 cannot be viewed from the opening 3) (refer to FIG. 1B and FIG. 2B). When the opening 3 and the opening 12 overlap each other, the vitreous body V is drawn into a suction path (aspiration passage) 13 in the inner cylindrical blade 10.

The outer cylindrical blade 1 is formed into a hollow cylindrical shape having an outer diameter of about 0.7 mm to about 1.5 mm and a thickness of about 0.05 mm to 0.2 mm (an inner diameter of about 0.6 mm to about 1.1 mm). Further, a distal end thereof is formed with a curved (rounded) surface 2. The reason for this is that consideration is taken for infliction of damage on a retina, which would otherwise be caused when the distal end of the outer cylindrical blade 1 comes into contact with the retina. As a matter of course, it may be a case that the curved surface is not formed.

An opening 3 is formed at the distal end of the outer cylindrical blade 1 by cutting a portion of the curved surface 2. This opening 3 is a suction (aspiration) hole for drawing the vitreous body V into the outer cylindrical blade 1 by suction (aspiration), and an edge 3 on an inner-wall-side of the opening 3 acts as an outer blade. The shape of the inner wall of the outer cylindrical blade 1 essentially coincides with the shape of an outer wall of an inner cylindrical blade 10 to be described later.

The inner cylindrical blade 10 is fitted into the outer cylindrical blade 1 and is held so as to be rotatable about a center axis thereof. The inner cylindrical blade 10 is formed into a hollow cylindrical shape having an outer diameter essentially coincides with the inner diameter of the outer cylindrical blade 1 (a clearance of micrometers to tens of micrometers exists between the inner wall of the outer cylindrical blade 1 and the outer wall of the inner cylindrical blade 10). A curved (round) surface 11 substantially coinciding with the inner wall of the curved surface 2 of the distal end of the outer cylindrical blade 1 is formed at a distal end of the inner cylindrical blade 10.

An opening 12 is formed at the distal end of the inner cylindrical blade 10 by cutting a portion of the curved surface 11. This opening 12 is a suction (aspiration) hole for drawing the vitreous body V into the inner cylindrical blade 10 by suction (aspiration), and an edge 12a on the outer wall-side of the opening 12 acts as an inner blade.

When the opening 3 and the opening 12 overlap each other by rotating the inner cylindrical blade 10 within the outer cylindrical blade 1, the vitreous body V is drawn into the suction path 13 in the inner cylindrical path 10 by suction. When the opening 12 and the opening 3 do not overlap each other by further rotating the inner cylindrical blade 10 within the outer cylindrical blade 1, the vitreous body V is cut by meshing engagement between the edge 3a and the edge 12a.

The outer cylindrical blade 1 is fixed to a cover 21 of a cutter main body 20a. A lead screw 10a is fixed to a substantial middle portion (which is disposed in the main body 20a) of the inner cylindrical blade 10 fitted into the outer cylindrical blade 1. The lead screw 10a is brought in mesh with a nut 22, the nut 22 is fixed to a moving base 24, and the moving base 24 is fixed to a diaphragm 23 arranged in the mainbody 20a. A material of the lead screw 10a is, for example, stainless steel. A material of the nut 22 is, for example, resin (polyimide) having poor affinity with the lead screw 10a. Friction resistance in converting linear reciprocating movement into rotational reciprocating movement is made to be reduced by combining the lead screw and the nut having different properties of surfaces thereof.

An outer peripheral portion of the diaphragm 23 is pinched between and held by a cover 25 and a cover 26 of the main body 20a. The moving base 24 is fixed to the nut 22 through a hole 25b opened at a partition 25a of the cover 25. The cover 26 is formed with nipples 26a and 26c, an opening 26b of the nipple 26a is communicated with a gas chamber 31 formed by the diaphragm 23 and an inner wall of the cover 26, and an opening 26d of the nipple 26c is communicated with the suction path 13 in the inner cylindrical blade 10. An outer peripheral portion of a substantial rear end portion of the inner cylindrical blade 10 is sealed from the diaphragm 23 and the cover 26 by O rings 30 to maintain airtightness of the suction path 13 and the gas chamber 31.

When compressed gas (compressed air) is supplied to the gas chamber 31 via the nipple 26a, the diaphragm 23 is linearly advanced (linearly moved forward) to a distal end side of the cutter 20 (the outer cylindrical blade 1 and the inner cylindrical blade 10) and the nut 22 fixed to the moving base 24 is linearly advanced in accordance therewith. By advancement of the nut 22, the lead screw 10a and the inner cylindrical blade 10 fixed thereto are rotated about the center axis.

Further, a compression spring 27 is provided between the lead screw 10a and the partition 25a, and the lead screw 10a and the inner cylindrical blade 10 are urged to the distal end side by spring force of the spring 27. Further, a helical torsion spring 28 fixed to the cover 21 is fixed o the lead screw 10a. The spring 28 is provided with urge force for rotating the lead screw 10a in a direction reverse to the direction of rotating the lead screw 10a by the advancement of the nut 22. An outer peripheral portion of the nut 22 is fixed with blocks 29 at two locations thereof for preventing the nut 22 from rotating, and the blocks 29 are slid in grooves 21a in the cover 21. Holes 21b and 21c are holes for passing gas in accordance with movement of the nut 22.

Further, a relationship between the nut and the lead screw for converting the linear reciprocating movement into the rotational reciprocating movement may be reversed. That is, the lead screw may linearly be reciprocated and the nut fixed to the inner cylindrical blade 10 may rotationally be reciprocated.

The nipple 26a communicated with the gas chamber 31 is communicated with an electromagnetic valve 105 of an operating unit 100 of the surgical equipment via a tube 42, and the electromagnetic valve 105 is communicated with a compressing pump 106 for delivering the compressed gas. Further, the nipple 26c communicated with the suction path 13 is communicated with a suction pump 102 of the operating unit 100 via a tube 41, and the pump 102 is communicated with an abandon chamber 101. The electromagnetic valve 105, the pump 106 and the pump 102 are controlled to be driven by a control portion 110 of the operating unit 100. Further, the control portion 110 is connected with a setting panel 107 for setting surgical conditions, and a foot switch 108 for inputting signals for operating the electromagnetic valve 105, the pump 106 and the pump 102.

Operation of the vitreous body surgical equipment having such a configuration will now be described. First, with the switches of the setting panel 107, surgical conditions (e.g., suction pressure, a cutting speed of the cutter 20, and the like) is set. Next, a perfusion (irrigation) liquid from an unillustrated perfusion liquid bottle is introduced into an eye of a patient. Moreover, the outer cylindrical blade 1 is inserted into the eye such that the opening 3 is situated a diseased area, such as an opaque are. Subsequently, the foot switch 108 is stepped on, to thus activate the pump 106 and electromagnetic valve 105 at a preset cutting speed and activate the pump 102 at preset suction (aspiration) pressure.

When the electromagnetic valve 105 is opened, the compressed gas is supplied from the pump 106 to the gas chamber 31 via the tube 42 and the nipple 26a. Thereby, inner pressure of the gas chamber 31 is increased, the diaphragm 23 is linearly advanced against the spring force of the spring 28, and the nut 22 is pressed to the distal end side via the moving base 24. Since the nut 22 is prevented from rotating by the blocks 29, the nut 22 is linearly advanced to the distal end side while rotating the lead screw 10a. The inner cylindrical blade 10 is rotated (regular rotation) by the rotation of the lead screw 10a.

When the electromagnetic valve 105 is closed, the gas chamber 31 is opened (communicated with the atmosphere) and the gas in the gas chamber 31 is delivered out. Thereby, the inner pressure of the gas chamber 31 is reduced, the lead screw 10a and the inner cylindrical blade 10 are rotated in a direction reverse to the previous direction by the spring force of the spring 28, and the nut 22 is pressed back and linearly retreated (linearly moved backward) in the direction reverse to the previous direction. The moving base 24 and the diaphragm 23 is pressed back and linearly retreated by the treat of the nut 22. The inner cylindrical blade 10 is rotationally reciprocated by controlling the opening/closing of the electromagnetic valve 105 by the control portion 110.

Further, instead of reversely rotating the inner cylindrical blade 10 by the spring force of the spring 28, the inner cylindrical blade 10 may be reversely rotated by sucking the gas in the gas chamber 31 by providing a suction pump at a portion of the electromagnetic valve 105 communicated with the atmosphere. Further, the cutting speed can further be accelerated by utilizing both of the spring force of the spring 28 and negative pressure of the suction pump.

The rotational reciprocation of the inner cylindrical blade 10 is for preventing involvement of the vitreous body V. When the inner cylindrical body 10 rotates about the center axis and the opening 3 and the opening 12 overlap each other, the vitreous body V is drawn into the suction path 13 by suction through the opening 3 and the opening 12. When the inner cylindrical blade 10 rotates further, and the opening 3 and the opening 12 do not overlap each other, the vitreous body B is cut by meshing engagement between the edge 3a and the edge 12a. The cut vitreous body V is sucked by the suction force by the pump 102 and is discharged to the abandon chamber 101 via the tube 41. By rotationally reciprocating the inner cylindrical blade 10, a time period of overlapping the opening 3 and the opening 12 can be set to be longer than that in the case of the one-directional rotation and efficiency of drawing the vitreous body V by suction can be increased.

Further, the inner cylindrical blade 10 of the cutter 20 is rotationally reciprocated by a single drive source (a source of making compressed gas flow in and out) and therefore, the inner cylindrical blade can be used with exchanging it for a vitreous body cutter of a guillotine type for linearly reciprocating the inner cylindrical blade. Further, by branching the tube 42 connected to the electromagnetic valve 105 by a three way plug and connecting the branched tube 42 to the vitreous body cutter of the guillotine type, both of the rotary type vitreous body cutter 20 and the guillotine type vitreous body cutter can be used. For example, the vitreous body at a center portion in the eye is cut by the vitreous body cutter of the guillotine type having excellent cutting efficiency and the vitreous body at a vicinity of the retina is cut by the vitreous body cutter 20 of the rotary type and so on.

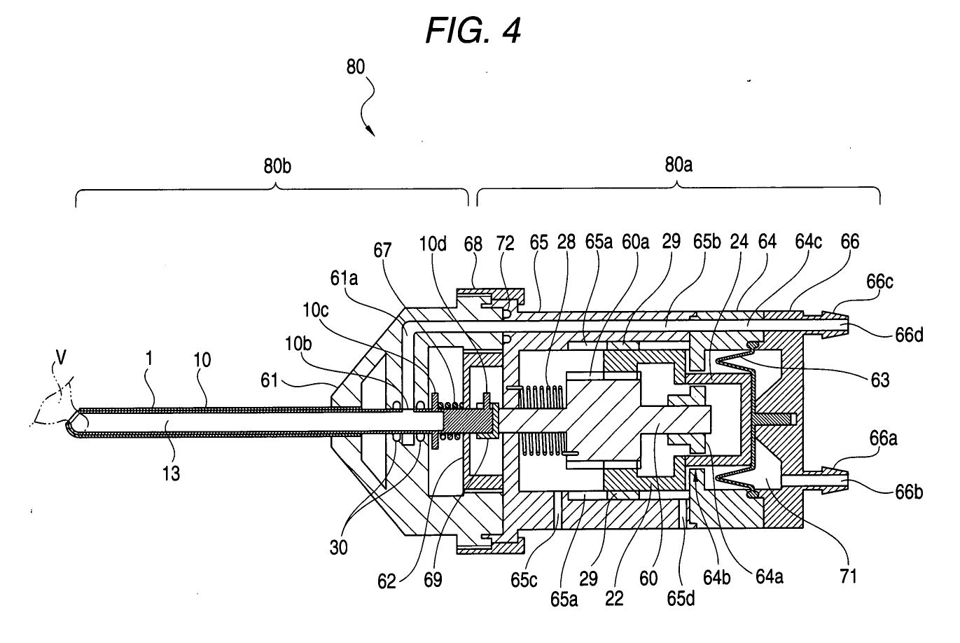

FIG. 4 is a schematic constitution view showing a rotary type vitreous body cutter according to another embodiment. Although the vitreous body cutter is basically disposable, the vitreous body cutter of FIG. 4 is constructed by a construction in which by making a blade side and a drive mechanism side of the vitreous body cutter separable, only the blade side is disposable and the drive mechanism side is reused. In FIG. 4, members the same as those of the above-described embodiment are attached with the same notations and detailed explanation thereof will be omitted.

A vitreous body cutter 80 is constituted by a drive mechanism portion 80a and a blade portion 80b separable therefrom. The outer cylindrical blade 1 is fixed to a cover 61 of the blade portion 80b, and the inner cylindrical blade 10 is held to be rotatable about the center axis by O rings 30 fixed to the cover 61 and the outer cylindrical blade 1. A side surface of the inner cylindrical blade 10 is formed with a hole 10b and a flange 10c. A compression coil spring 67 is provided ay the cover 61 by a lid 62 screwed to the cover 61, and the inner cylindrical blade 10 is urged to the distal end side via the flange 10c by spring force of the spring 67. Further, a rear end portion of the inner cylindrical blade 10 is formed with a projected portion 10d.

The cover 61 and a cover 65 can be coupled by a screw 68. A cover 64 is fixed to the cover 65 and a cover 66 is fixed to the cover 64. A shaft 60 having a lead screw 60a is axially supported by a partition 64a of the cover 64. The lead screw 10a is brought in mesh with the nut 22, the nut 22 is fixed to the moving base 24, and the moving base 24 is fixed to a diaphragm 63. An outer peripheral portion of the diaphragm 63 is pinched between and held by the cover 64 and the cover 66. The moving base 24 is fixed to the nut 22 via a hole 64b opened at the partition 64a of the cover 64. The helical torsion spring 28 fixed to the cover 65 is fixed to the lead screw 60a. The spring 28 is provided with urge force of rotating the lead screw 60a in a direction reverse to a direction of rotating the lead screw 60a by advancement of forward the nut 22. The outer peripheral portion of the nut 22 is fixed with the blocks 29 for preventing rotation of the nut 22 at the two locations and the blocks 29 are slid in grooves 65a in the cover 65. Holes 65c and 65d are holes for passing gas in accordance with movement of the nut 22. A distal end of the shaft 60 is fixed with a fitting portion 69 and the fitting portion 69 is fitted with the projected portion 10d of the inner cylindrical blade 10 to transmit rotation of the shaft 60 to the inner cylindrical blade 10. That is, the fitting portion 69 and the projected portion 10d constitute a transmitting mechanism for transmitting rotational reciprocating movement of the shaft 60.

The cover 66 is formed with nipples 66a and 66c. An opening 66b of the nipple 66a is communicated with a gas chamber 71 formed by the diaphragm 63 and an inner wall of the cover 66. An opening 66d of the nipple 66c is communicated with the suction path 13 in the inner cylinder portion 10 via a suction path 64c in the cover 64, a suction path 65b in the cover 65, a suction path 61a at inside of the cover 61, and the hole 10b of the inner cylindrical blade 10. O rings 72 are fitted between the suction path 65b of the cover 65 and the suction path 61a of the cover 61 in order to maintain airtightness.

The nipple 66a communicated with the gas chamber 71 is communicated with the electromagnetic valve 105 via the tube 42. The nipple 66c communicated with the suction path 13 is communicated with the pump 102 via the tube 41.

When compressed gas is supplied to the gas chamber 71, inner pressure of the gas chamber 71 is increased, the diaphragm 63 is linearly advanced against the spring force of the spring 28, and the nut 22 is pressed to the distal end side via the moving base 24. Since the nut 22 is prevented from rotating by the blocks 29, the nut 22 is linearly advanced to the distal end side while rotating the lead screw 60a. The inner cylindrical blade 10 is rotated (regularly rotated) by the rotation of the lead screw 60.

The gas chamber 71 is opened (communicated with the atmosphere), the gas in the gas chamber 71 is delivered out, the inner pressure of the gas chamber 71 is reduced, the lead screw 60a is rotated in a direction reverse to a previous direction by the spring force of the spring 28, the nut 22 is pressed back and linearly retreated in the direction reverse to the distal end side. The moving base 24 and the diaphragm 63 are pressed back and linearly retreated by retreat of the nut 22. Further, by reversely rotating the lead screw 60a, also the inner cylindrical blade 10 is rotated reversely.

According to the vitreous body cutter 80, by loosening the screw 68, the blade portion 80b can be separated from the drive mechanism portion 80a and only the blade portion 80b can be interchanged.

What is claimed is:

1. A vitreous body cutter for cutting a vitreous body in an eye, the vitreous body cutter comprising:
an inner cylindrical blade having a first suction hole and a suction path;
an outer cylindrical blade having a second suction hole, the outer cylindrical blade holding the inner cylindrical blade to be rotatable about a center axis thereof;
a main body fixed with the outer cylindrical blade;
a diaphragm arranged in the main body, the diaphragm being linearly advanced to a distal end side of the blades in a direction of the center axis by supplying compressed gas into a gas chamber formed by the diaphragm and the main body; and
a converting-and-transmitting mechanism which converts linearly advancement of the diaphragm into a rotation about the center axis and transmits the rotation to the inner cylindrical blade to rotate the inner cylindrical blade about the center axis.

2. The vitreous body cutter according to claim 1, wherein
the converting-and-transmitting mechanism includes a lead screw, a nut and a torsion spring, the lead screw and the nut being brought in mesh with each other,
one of the lead screw and the nut is fixed to the inner cylindrical blade,
the other of the lead screw and the nut is linearly advanced by the diaphragm being linearly advanced against spring force of the torsion spring by supplying the compressed gas into the gas chamber to rotate the one in a first direction, and
the other is linearly retreated by the diaphragm being linearly retreated by the spring force of the torsion spring by releasing the gas from the gas chamber to rotate the one in a second direction opposed to the first direction.

3. The vitreous body cutter according to claim 1, wherein
the converting-and-transmitting mechanism includes a lead screw and a nut, the lead screw and the nut being brought in mesh with each other,
one of the lead screw and the nut is fixed to the inner cylindrical blade,
the other of the lead screw and the nut is linearly advanced by the diaphragm being linearly advanced by supplying the compressed gas into the gas chamber to rotate the one in a first direction, and
the other is linearly retreated by the diaphragm being linearly retreated by sucking the gas from the gas chamber to rotate the one in a second direction opposed to the first direction.

4. The vitreous body cutter according to claim 1, wherein
the main body includes a blade portion fixed with the outer cylindrical blade holding the inner cylindrical blade, and a drive mechanism portion provided with the diaphragm and the converting-and-transmitting mechanism, and
the blade portion and the drive mechanism portion are separable from each other.

5. The vitreous body cutter according to claim 4, further comprising a transmitting mechanism provided at the blade portion, the transmitting mechanism transmitting the rotation converted by the converting-and-transmitting mechanism to the inner cylindrical blade.

6. A vitreous body surgical equipment comprising:
a vitreous body cutter according to claim 1;
a gas supply unit which supplies the compressed gas into the gas chamber of the vitreous body cutter;
a control unit which controls to supply the compressed gas; and
a suction unit which generates suction pressure in the suction path in the inner cylindrical blade.
